SEQUENCE LISTING (1 sequence)

SEQ ID NO: 1 - DNA, 5017 bp, human
  promoter (1)..(800): SCA2 promoter
ggtacccagatgtgggtgggccccggagggttgttccactcccagccccggcagggcaggacagcgcggcctgcctggtagatgccccgagccactggagcgcctactgtgtggcgggcgggggacggcaggaaaacggcaggatgctgtgtcccctgaatctggcagggttctaggtgctttacacgtagcaagacacattctccgccccaaggcactcgcagtcagtccattttctgggttgcatcaggtgggggcaaactaggtccccgcagaagtgaagatgctgaaggaatacagtaggagaagaaatgttctctcctgtccttccacaccaggcaggcccagacggctgagaccgacacgccctccccgaagggcagacccgcctcgaggaaggcggatccgggtagggaccgccgctggccctcacccgacccccgagaaagcaacccagcgcgccgcccgctcctcacgtgtccctcccggccccggggccacctcacgttctgcttccgtctgacccctccgacttccggtaaagagtccctatccgcacctccgctcccacccggcgcctcggcgcgcccgccctccgatgcgctcagcggccgcagctcctcggagtcccgcggtggccaccgagtctcgccgcttcgccgcagccaggtggcccgggtggcgctcgctccagcggccggcgcggcggagcgggcggggcggcggtggcgcggccccgggaccgtatccctccgccgcccctcccccgcccggccccggcccccctccctcccggcagagctcgcctccctccgcctcagactgttttggtagcaacggcaacggcggcggcgcgtttcggcccggctcccggcggctccttggtctcggcgggcctccccgccccttcgtcgtcgtccttctccccctcgccagcccgggcgcccctccggccgcgccaacccgcgcctccccgctcggcgcccgtgcgtccccgccgcgttccggcgtctccttggcgcgcccggctcccggctgtccccgcccggcgtgcgagccggtgtatgggcccctcaccatgtcgctgaagccccagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagcagccgccgcccgcggctgccaatgtccgcaagcccggcggcagcggccttctagcgtcgcccgccgccgcgccttcgccgtcctcgtcctcggtctcctcgtcctcggccacggctccctcctcggtggtcgcggcgacctccggcggcgggaggcccggcctgggcagaggtcgaaacagtaacaaaggactgcctcagtctacgatttcttttgatggaatctatgcaaatatgaggatggttcatatacttacatcagttgttggctccaaatgtgaagtacaagtgaaaaatggaggtatatatgaaggagtttttaaaacttacagtccgaagtgtgatttggtacttgatgccgcacatgagaaaagtacagaatccagttcggggccgaaacgtgaagaaataatggagagtattttgttcaaatgttcagactttgttgtggtacagtttaaagatatggactccagttatgcaaaaagagatgcttttactgactctgctatcagtgctaaagtgaatggcgaacacaaagagaaggacctggagccctgggatgcaggtgaactcacagccaatgaggaacttgaggctttggaaaatgacgtatctaatggatgggatcccaatgatatgtttcgatataatgaagaaaattatggtgtagtgtctacgtatgatagcagtttatcttcgtatacagtgcccttagaaagagataactcagaagaatttttaaaacgggaagcaagggcaaaccagttagcagaagaaattgagtcaagtgcccagtacaaagctcgagtggccctggaaaatgatgataggagtgaggaagaaaaatacacagcagttcagagaaattccagtgaacgtgaggggcacagcataaacactagggaaaataaatatattcctcctggacaaagaaatagagaagtcatatcctggggaagtgggagacagaattcaccgcgtatgggccagcctggatcgggctccatgccatcaagatccacttctcacacttcagatttcaacccgaattctggttcagaccaaagagtagttaatggaggtgttccctggccatcgccttgcccatctccttcctctcgcccaccttctcgctaccagtcaggtcccaactctcttccacctcgggcagccacccctacacggccgccctccaggcccccctcgcggccatccagacccccgtctcacccctctgctcatggttctccagctcctgtctctactatgcctaaacgcatgtcttcagaagggcctccaaggatgtccccaaaggcccagcgacatcctcgaaatcacagagtttctgctgggaggggttccatatccagtggcctagaatttgtatcccacaacccacccagtgaagcagctactcctccagtagcaaggaccagtccctcggggggaacgtggtcatcagtggtcagtggggttccaagattatcccctaaaactcatagacccaggtctcccagacagaacagtattggaaatacccccagtgggccagttcttgcttctccccaagctggtattattccaactgaagctgttgccatgcctattccagctgcatctcctacgcctgctagtcctgcatcgaacagagctgttaccccttctagtgaggctaaagattccaggcttcaagatcagaggcagaactctcctgcagggaataaagaaaatattaaacccaatgaaacatcacctagcttctcaaaagctgaaaacaaaggtatatcaccagttgtttctgaacatagaaaacagattgatgatttaaagaaatttaagaatgattttaggttacagccaagttctacttctgaatctatggatcaactactaaacaaaaatagagagggagaaaaatcaagagatttgatcaaagacaaaattgaaccaagtgctaaggattctttcattgaaaatagcagcagcaactgtaccagtggcagcagcaagccgaatagccccagcatttccccttcaatacttagtaacacggagcacaagaggggacctgaggtcacttcccaaggggttcagacttccagcccagcatgtaaacaagagaaagacgataaggaagagaagaaagacgcagctgagcaagttaggaaatcaacattgaatcccaatgcaaaggagttcaacccacgttccttctctcagccaaagccttctactaccccaacttcacctcggcctcaagcacaacctagcccatctatggtgggtcatcaacagccaactccagtttatactcagcctgtttgttttgcaccaaatatgatgtatccagtcccagtgagcccaggcgtgcaacctttatacccaatacctatgacgcccatgccagtgaatcaagccaagacatatagagcagtaccaaatatgccccaacagcggcaagaccagcatcatcagagtgccatgatgcacccagcgtcagcagcgggcccaccgattgcagccaccccaccagcttactccacgcaatatgttgcctacagtcctcagcagttcccaaatcagccccttgttcagcatgtgccacattatcagtctcagcatcctcatgtctatagtcctgtaatacagggtaatgctagaatgatggcaccaccaacacacgcccagcctggtttagtatcttcttcagcaactcagtacggggctcatgagcagacgcatgcgatgtatgcatgtcccaaattaccatacaacaaggagacaagcccttctttctactttgccatttccacgggctcccttgctcagcagtatgcgcaccctaacgctaccctgcacccacatactccacaccctcagccttcagctacccccactggacagcagcaaagccaacatggtggaagtcatcctgcacccagtcctgttcagcaccatcagcaccaggccgcccaggctctccatctggccagtccacagcagcagtcagccatttaccacgcggggcttgcgccaactccaccctccatgacacctgcctccaacacgcagtcgccacagaatagtttcccagcagcacaacagactgtctttacgatccatccttctcacgttcagccggcgtataccaacccaccccacatggcccacgtacctcaggctcatgtacagtcaggaatggttccttctcatccaactgcccatgcgccaatgatgctaatgacgacacagccacccggcggtccccaggccgccctcgctcaaagtgcactacagcccattccagtctcgacaacagcgcatttcccctatatgacgcacccttcagtacaagcccaccaccaacagcagttgtaaggctgccctggaggaaccgaaaggccaaattccctcctcccttctactgcttctaccaactggaagcacagaaaactagaatttcatttattttgtttttaaaatatatatgttgatttcttgtaacatccaataggaatgctaacagttcacttgcagtggaagatacttggaccgagtagaggcatttaggaacttgggggctattccataattccatatgctgtttcagagtcccgcaggtaccccagctctgcttgccgaaactggaagttatttattttttaataacccttgaaagtcatgaacacatcagctagc

(19) United States
(12) Patent Application Publication
(10) Pub. No.: US 2006/0130165 A1
(43) Pub. Date: Jun. 15, 2006

(54) F066 transgenic mice that express pathogenic mutants of the sca2 gene

(76) Inventors: Jorge Agustin Aguiar Santiago, Ciudad de la Habana (CU); Julio Raul Fernandez Masso, Playa C. de la Habana (CU); Yssel Mendoza Mari, Ciudad de la Habana (CU); Maria Mercedes Vazquez Marcos, Ciudad de la Habana (CU); Silian Cruz Leon, C. de la Habana (CU); Luis Clodoaldo Velazquez Perez, Rpto Santiesteban (CU); Luis Saturnino Herrera Martinez, Ciudad de la Habana (CU)

Correspondence Address: HOFFMANN & BARON, LLP, 6900 JERICHO TURNPIKE, SYOSSET, NY, 11791, US

(21) Appl. No.: 11/297,660

(22) Filed: Dec. 8, 2005

(30) Foreign Application Priority Data
Dec. 9, 2004 (CU) CU2004/0278

(51) Int. Cl. A01K 67/027; C12N 5/06

(52) U.S. Cl. 800/18; 435/354

(57) ABSTRACT

The present invention is related with the neurobiology and the transgenic animals, more specifically with obtaining a model to study the SCA2 genetic disease. The genome of one mice line has been modified through the introduction of a DNA segment that contains the necessary information for the synthesis of the human ataxin 2. In particular the invention is related with the transgenic mice F066 and the cellular lines able to express the human sca2 gene under the regulation of its self promoter. The F066 transgenic mice reproduce the features of the disease. The homozygous transgenic mouse line for the new gene was obtained through matting. This transgenic line is useful to study the mechanism that produce the disease and also will permit to prove new therapeutics that contributes to minimize the clinical symptoms in the patients.

This application asserts the priority of Cuban application no. CU 2004/0278 filed on Dec. 9, 2004. The entire specification of Cuban application no. CU 2004/0278 is hereby incorporated by reference.

### FIELD OF THE INVENTION

The present invention is related with the neurobiology and the transgenic animals, more specifically with the obtaining and the methods to use an animal model to study the genetic disease spinocerebellar ataxia type 2 (SCA2). Its essence consist in that the genome of one mice line has been modified through the introduction of a deoxyribonucleic acid (DNA) segment that contain the necessary information for the synthesis of the human ataxin 2. In particular the invention is related with the transgenic mice F066 and the self derived cellular lines that will be able to express the human sca2 gene under the regulation of its self promoter. Those transgenic mice, named F066, reproduce some of the symptoms of the SCA2 disease.

### BACKGROUND OF THE INVENTION

The dominant spinocerebellar ataxias are a neurodegenerative disease group that affects mainly the Purkinje neurons of the cerebellum. The spinocerebellar ataxia type 2 (SCA2) is an autosomic dominant genetic disease characterized by the gait ataxia, difficulties in speech, abnormal behavior of the reflexes, and slow and limited eye movements (Velazquez L, Medina H E (1999). Neurophysiologic evaluation in patients affected by spinocerebellar ataxia type 2. Rev. Neurol 27 (160): 921-926).

Molecularly, the appearance of the symptoms of the disease its produce when the pathogenic allele present 32 or more CAG repeats, and these repeats are considered as expansions (Santos et al (1999). Molecular diagnosis of a sample of the Cuban population with spinocerebellar ataxia type 2. Biotecnologia Aplicada 16: 219-221). The beginning of the disease symptoms correlate inversely to the length of the expansion. The disease is progressive and patients die 15-18 years after symptom onset. The more frequently pathogenic repeat is 37 CAG or glutamines. The number of glutamine repeats is associated with the age of clinic symptoms onset from 20 to 60 years (Santos et al (1999). Molecular diagnosis of a sample of the Cuban population with spinocerebellar ataxia type 2. Biotecnologia Aplicada 16: 219-221).

The neuropathologic studies in death SCA2 patients show a marked reduction in the Purkinje cells number with poor dendrite arborization. There is also a decrease in the number of granule cells, the inferior olive neurons and the neurons from the pontocerebellar nuclei. There is also, a marked reduction of the neurons from the nigral substance, desmienilization of the spinal cord and reduction of the number and size of the motor neurons (Estrada R et al (1999) Spinocerebellar ataxia 2 (SCA2): morphometric analyses in 11 autopsies. Acta Neuropathol (Berl) 97(3): 306-310)

The molecular studies have been demonstrated that normal ataxin 2 is a 140 kDa protein ubiquitously expressed in the human and mice tissues. The messenger RNA in humans is localized in the brain, heart, placenta, liver, muscle and pancreas with little expression in kidney and lung. Different to other polyglutamine diseases the formation of nuclear aggregates is not associated with the pathogenic mechanism (Pulst et al (1996). Moderate expansion of a normally biallelic trinucleotide repeat in spinocerebellar ataxia type 2. Nature Genet 14: 269-276).

The degenerative process of Huntington and SCA diseases are very difficult to elucidate and very aggressive. The identification of the mutant genes in these diseases has permitted the determination of the pathogenesis to a basic level. Recently, studies in transgenic mouse models and patients have permitted to know some of the molecular changes that conduce to neuronal dysfunction and death in the polyglutamine diseases. At the same time, the appearance of mutant mice that show neurological phenotypes have permitted to discover other type of secondary neurodegenerative diseases like the alteration in the ionic canals or the alteration in the neurotransmitter receptor functions (Heintz N, Zoghbi Y (2000). Insights from mouse models into the molecular basis of neurodegeneration, Annu. Rev. Physiol 62: 779-802).

Although the studies in normal and disease human brains could bring important aspects to the knowledge of the pathogenesis of the diseases caused by polyglutamine expansions, some observations are limited to the terminal steadies of the disease. The transgenic models could circumvent this problem, but many of then are constructed using truncated constructs with large polyglutamine tracts to produce neurodegeneration. Beside, a great number of these models don't show high neuronal loss, feature that define the polyglutamine diseases in humans (Davies et al 1999). From neuronal inclusions to neurodegeneration: neuropatological investigation of a transgenic mouse model of Huntington's disease. Phil. Trans. R. Soc. Lond. B Biol. Sci. 234: 981-989).

In the U.S. Pat. No. 6,515,197 it is describe a transgenic SCA2 model developed by (Huynh et al (2000). Nuclear localization or inclusion body formation of ataxin-2 is not necessary for SCA2 pathogenesis in mouse or human, Nature Genetics 26: 1-7). These animals show ataxin 2 accumulation in the cytoplasm and symptoms of the SCA2 disease, but its limitations are the transgene expression and the pathogenic protein production only in the Purkinje cells. Also, the relatively few number of CAG/polyglutamine repeats of the pathogenic allele (58 CAG-58 Q) from these transgenic mice produce deficiencies in the study of the protein effects in the cell lines derived of this model, because the cells don't show clear phenotypic effects.

In consequence, in this field it's imposing the expression of the SCA2 gene regulated under its self promoter. The transgenic animals generated with this construction will express the heterologous gene not only in the Purkinje cell, also will simulate the expression patterns of the SCA2 gene in other cells and tissues like occur in humans. Additionally, the use of a larger expansion (75 CAG-75 Q) brings more possibilities of the obtaining of cell lines derived of the transgenic mice with evident phenotypic effects.

### DETAILED DESCRIPTION OF THE INVENTION

The present invention show a useful transgenic mouse model to study the genetic disease spinocerebellar ataxia type 2, where the mouse it's characterize by the human SCA2 gene expression regulated by it's self promoter, and also it's capable of reproduce some of the features of the human disease. The transgenic mice could be homozygous or heterozygous for the transgene.

It is a general objective of the present invention to produce transgenic mice or cell lines derived from them, that contain one or two copies of the human SCA2 gene with 75 CAG repeats integrated in the genome.

An aspect of the present invention is to proportionate a method for the screening of different biological or chemical agents capable to modulate the associated phenomena with the neurodegeneration, for example the motor in-coordination, the apoptosis and the formation of polyglutamine aggregates, where the method involucrate the steps to combine a candidate substance with the transgenic mouse and the evaluation of his effects in the model. These substances could be short chemical molecules or endogenous or exogenous biological molecules useful for the prevention, the treatment, or the diminution of the clinical symptoms of the disease. Another objective is to proportionate a murine model of the study of the genetic disease spinocerebellar ataxia type 2, that permit the study of the progress of the disease and the associated symptoms, and the identification of the prevention mediums.

In another aspect of the invention, the transgenic mice could be additionally manipulate to permit the expression of other genes that could have therapeutic effects like for example growth factors like IGF-1, NGF, BDNF, and anti-apoptotic genes like Bcl-2 and others.

Transgenic Animals

In the present invention the exogenous gene is united operationally to the minimal promoter region of the human SCA2 gene (FIG. 1). This promoter is functional in several cellular lines of different origin and specie (Aguiar et al (1999). Cloning and sequence of the 5′ region from human spinocerebellar ataxia 2 gene. Biotecnologia Aplicada 16: 165-168, Aguiar et al (1999). Identificaction of the physiological promoter for spinocerebellar ataxia type 2 reveals a CpG island for promoter activity situated into the exon 1 of this gene and provides data about the origin of the nonmethylated state of these types of islands, Biochem. Biophys. Res. Commun. 254: 315-318).

The transgene contains a pathological expansion of 75 CAG repeats, one of the largest reported for a Cuban patient. This patient has a disease onset at 6 years old. The gene could be manipulate with the objective of introduce deletions, substitutions or insertions like for example to increase the number of CAG repeats. The promoter region could be modified with the objective to regulate or increase the level of the SCA2 gene expression with pathological CAG repeats. The transgenic mouse of the present invention could receive other alterations like for example to eliminate the function of the endogenous gene.

Nuclei Acid Composition

The constructions for his use in the present invention could include any construction appropriate for the generation of transgenic animals that express the SCA2 gene or fragments of this gene that contains 75 or more CAG repeats operated under the regulation of his self promoter in the different cells and tissues like occur in humans. These constructions could contain cDNA, genomic sequences or both.

The methods for the isolation and cloning of the target sequences from different sources and the constructions for his expression are reported in the literature. Besides, the constructions could contain reporter genes like Lac Z, under the control of the human SCA2 promoter to facilitate the detection of the expression. It is possible to generate protein fusion or other modifications that permit to make localization studies in tissues or sub-cellular regions. Other mutations could be use to make structure-function studies.

Other Human Genes

There are a number of human genes involved in neurodenegeration mechanisms. Transgenic animals in the present invention could carry additional genes that accelerate neurodegeneration processes, neuronal plasticity or mediate neuroprotection.

Drug Screening Assays

It is possible to identify ligands or substrates that mimic neurodegeneration processes using transgenic animals or their derived cell lines expressing pathogenic variants of sca2 gene. Such compounds could act either directly on ataxin 2 or on mechanisms associated to the disease. Thus, identified therapeutical targets and designed drugs derived from the study of these transgenic animals could be useful for other CNS pathologies since they share common neurodegenerative mechanisms, including insoluble aggregates formation and apoptosis activation. That is the case of low toxicity drugs screening.

A wide number of assays can be use in this way, for example motor behaviour evaluation, immunenzymatic and molecular biology assays, neurophysiological evaluation, and localization of drugs. Mice or mice-derived cells could be useful depending on the assay. Cells can be extracted from animals or immortalized. Central Nervous System derived cells are of particular interest, specifically Purkinje cells. Besides, other cell types, for example bone marrow stromal cells.

Therapeutical candidates include a group of chemical compounds, tipically low weight organic molecules, an also biomolecules, such as peptides, carbohydrates, fatty acids, steroids, lipids and their derivatives or combinations. Candidates can be obtained from different sources, including libraries containing large numbers of natural and synthetic compounds. Those compounds can be modify to produce structural analogs with improved pharmacological properties. Among therapeutical agents are bone marrow stromal cells, which can be derived from transgenic or wild type mice. These cells can be genetically engineered and systemically or stereotactically administered directly into the lession site. Screening can be managed to pharmacologically well known active compounds or their analogs or derivatives. To obtain therapeutical effects candidates can be formulated and administered with a pharmaceutically appropriate carrier.

High throughput in vivo screening usually includes the use of a number of animals receiving different concentrations of therapeutical candidate. It also includes different formulations of the same candidate. These agents can be administered in one dose only combined with other molecules when it is possible to obtain a synergic effect. Monitoring the effect ca be carried out by conventional methodologies.

### DESCRIPTION OF THE DRAWINGS

FIG. 1. Sequence of the sca2 human promoter fused to the sca2 human gene with 75 CAG repeats (total length of 5.017 kbp). In bold, restriction sites for KpnI (ggtacc) and Sacd (gagctc), flanking human sca2 gene promoter (0.8 kbp) (Aguiar et al. 1999. Biochem. Biophys. Res. Commun. 254: 315-318). Within sca2 cDNA sequence (4.217 kbp), in bold 75 CAG region, isolated from a Cuban patient.

FIG. 2. Schematic representation of DNA fragment for microinjection (5.285 kbp): human sca2 gene promoter (0.8 kbp), human sca2 cDNA (4.217 kbp) with 75 CAG repeats, SV40 polyA signal (0.268 kbp).

FIG. 3. Transgen construction design.

FIG. 4. Polymerase chain reaction on chromosomal DNA.

(A) 1-3: F0 sca2-75CAG transgenic mice; 4: wild type mouse; 5: molecular weight marker BlueScript/HpaII, 6: control plasmid for 22 CAG repeats, 7: control plasmid for 75 CAG repeats, 8: water as negative control.

(B) 1-6: F2 sca2-75CAG transgenic mice, 7: wild type mouse, 8: cuban patient carrying 22 and 41 CAG repeats, 9: control plasmid for 75 CAG repeats, 10: molecular weight marker BlueScript/HpaII.

FIG. 5. Southern blot.

(A) SacI-EcoRI fragment hybridization. 1-2: F0 sca2-75CAG transgenic mice, 3: wild type mouse, 4: 0.1 ng of control plasmid for 75 CAG repeats, Sacd-EcoRI digested.

(B) KpnI fragment hybridization. 1: 0.01 ng of control plasmid for 75 CAG repeats, KpnI digested, 2: wild type mouse, 3-7 F2 sca2-75CAG transgenic mice.

FIG. 6. Transgene expression analysis in different tissues of transgenic mice by Northern blot. Age-paired wild type animals as negative controls. Molecular weight for human sca2-75CAG transgene (4 kbp) is indicated by arrows.

(A) Cerebellum: 1-F2 transgenic mouse, homozygous for sca2-75CAG human gene. 2-F1 transgenic mouse, heterozygous for sca2-75CAG human gene. 3-Wild type mouse.

(B) 1, 3, 5-Brain, liver and skeletal muscle from wild type mouse. 2, 4, 6, 7, 8 -Brain, liver, skeletal muscle, lung and kidney from heterozygous transgenic mouse.

FIG. 7. Transgene expression analysis in different tissues of transgenic mice by Western blot. Age-paired wild type animals as negative controls. Western blots were carried out using a monoclonal antibody raised against the N-terminal fragment of the human ataxin 2 protein, which includes ATG and a 22 CAG repeats region. Arrows point to wild type ataxin 2 protein (140 KD). Higher molecular weight proteins could be expanded ataxin 2 protein with 75 CAG repeats or ataxin 2 protein aggregated to different proteins. In samples corresponding to transgenic mice there are strongly stained lower weight proteins, which could be degradation fragments of expanded ataxin 2 with 75 CAG repeats.

(A) 1-Monoclonal antibody (150 KD) as molecular weight marker. 2-Cerebellum from wild type mouse. 3-Cerebellum from heterozygous sca2-75CAG transgenic mouse.

(B) 1-Cerebellum from wild type mouse. 2-Monoclonal antibody (150 KD) as molecular weight marker. 3-9: Cerebellum, brain, liver, heart, skeletal muscle, kidney and lung from heterozygous sca2-75CAG transgenic mouse.

FIG. 8. Rotarod test's results from two sca2-75CAG transgenic mice and one age-paired wild type mouse. The graph shows average performance on Rotarod apparatus of 4 trials each day on 5 consecutive days.

(A) Six weeks. Homozygous mice show clear symptoms of motor impairment compared to wild type.

(B) Twelve weeks. Both, homozygous and heterozygous transgenic mice show motor impairment compared to wild type.

FIG. 9. Analysis of footprint patterns of wild type mouse (A) and heterozygous F0 transgenic mouse (B). Quantitative comparison of step length (C) and linearity of the movements (D).

FIG. 10. Clasping posture. The wild type mouse extends the back legs as a classical posture in order to steady itself (A). An heterozygous transgenic mouse binds the back legs demonstrating the feet-clasping posture when suspended by the tail (B).

FIG. 11. Anatomical changes evaluation. (A, B, C) Cresyl Violet staining. (D, E, F) Anti-calbindin 28K immunhistochemistry. (A, D) Cerebellum from one year old wild type mouse. (B, C, E, F) Cerebellum from one year old heterozygous sca2-75CAG transgenic mouse.

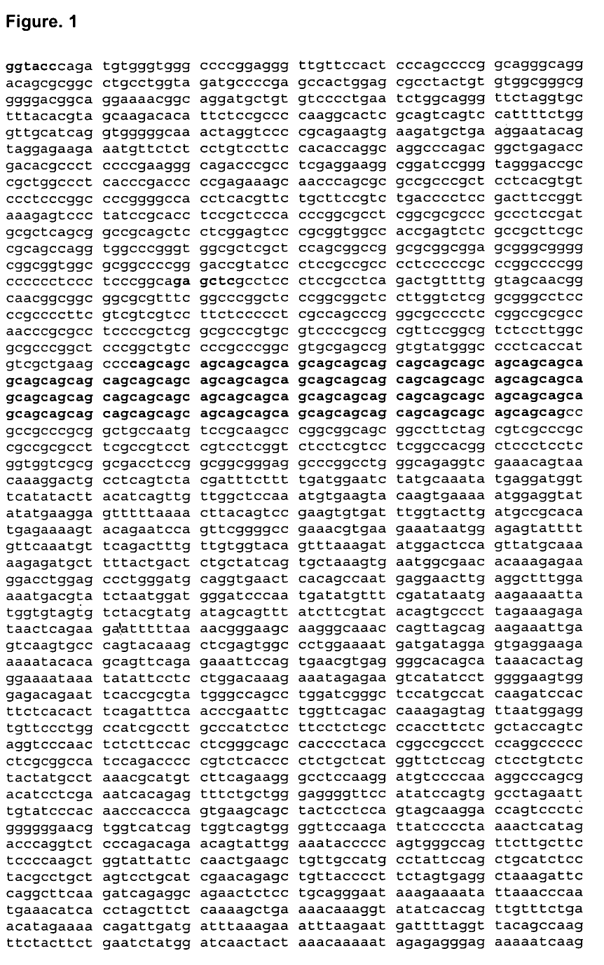

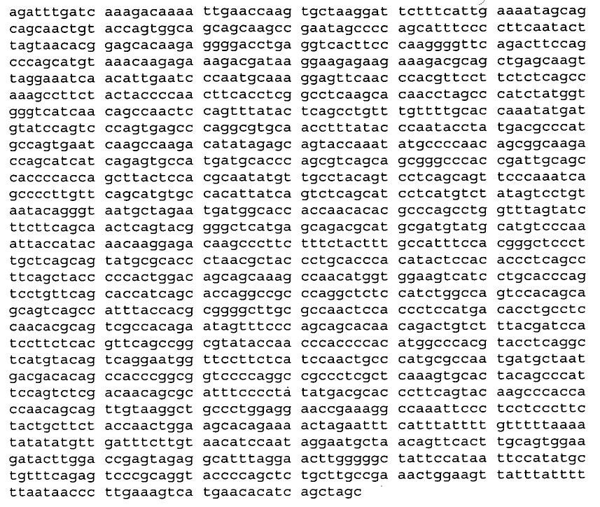

### EXAMPLES

### Example 1

### Obtention and Molecular Characterization of F0 Transgenic Mice Carrying sca2-75CAG Human Gene

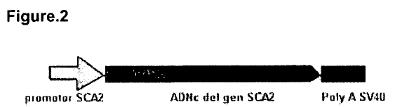

The genetic construction is composed by the following elements 5′-3′ ordered (FIG. 2):

a) A 0.8 kbp fragment encoding human ataxin 2 gene promoter (Aguiar et al (1999) Cloning and sequence of the 5′ region from human spinocerebellar ataxia 2 gene. Biotecnologia Aplicada 16: 165-168, Aguiar et al (1999). Identification of the physiological promoter for spinocerebellar ataxia type 2 reveals a CpG island for promoter activity situated into the exon 1 of this gene and provides data about the origin of the nonmethylated state of these types of islands, Biochem. Biophys. Res. Commun. 254: 315-318).

b) A 4.217 kbp fragment encoding complementary DNA for sca2 gene carrying 75 CAG repeats. This fragment was obtained from a patient blood total RNA sample by RT-PCR.

c) A 0.268 kbp fragment encoding transcription termination and polyadenylation signals from SV40 virus, present in pGL3-Luc Basic Vector (Promega) plasmid.

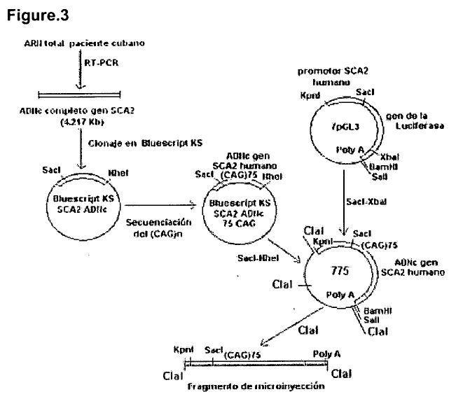

FIG. 3 schematically represents the genetic construction used to generate transgenic mice line. KpnI-SacI fragment containing sca2 gene promoter region was isolated from a genomic clone and was inserted into pGL3-Luc Basic Vector (Promega). The sca2 cDNA carrying 75 CAG repeats was isolated by RT-PCR from a cuban patient. This fragment was cloned into pBlueScript KS (Stratagene) plasmid.

The late expression genes 3′ region from SV40 virus carrying transcription termination and polyadenylation signals was obtained from pGL3-Luc Basic Vector (Promega) plasmid.

Final genetic construction was made following standard procedures described by Sambrook et al. (Sambrook et al (1982). Molecular cloning-a laboratory manual. 2nd Ed. Cold Spring Harbor Laboratory Press). Final plasmid was identified as 775. For transgenesis final construction was ClaI digested, to obtain the fragment carrying promoter, cDNA with 75 CAG repeats and SV40 signals encoding regions. Transgenic animals were obtained by previously described methods (Castro FO, Aguilar A (1989). Microinjection, culture, and transfer of one cell mouse embryos.

I-Obtention of one cell embryos and its culture. Interferón y Biotecnologia 6: 65-70, Castro F O, Aguilar A (1989). Microinjection, culture and transfer of one cell mouse embryos. II-Microinjection, culture and transfer of one cell mouse embryos (in Spanish). Interferón y Biotecnologia 6: 172-174, Castro F O, Aguilar A (1992). Effect of the number of transferred embryos microinjected or non-manipulated on the pregnancy rate and litter size in B6D2F1 mice. Theriogenology 36:105). Mice strain used was a hybrid B6D2F1 X OF1, obtained in our laboratory. Parent mice strains B6D2F1 and OF1 were obtained from National Center for the Production of Laboratory Animal, Havana, Cuba.

Transgenic mice were obtained by microinjection of 1-2 picolitres of buffer solution containing 2 ng per microliter linear DNA fragment into one of the unicellular embryo pronuclei of mouse. Embryos were transferred to recepter female mice and, after birth, littermates were analyzed to detect the presence of transgene by Polymerase Chain Reaction (PCR) (Saiki et al (1989). Primer directed amplification of DNA with a thermostable DNA polymerase. Science 239: 487-491).

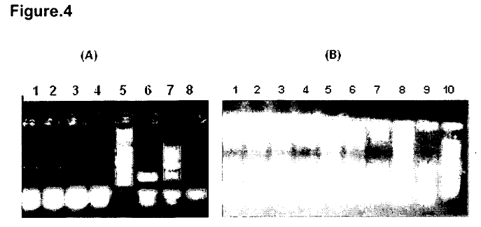

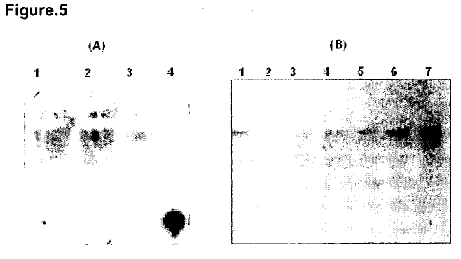

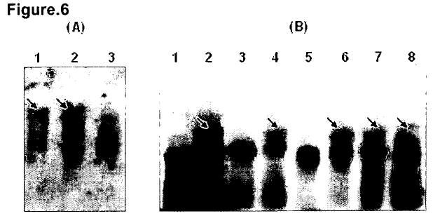

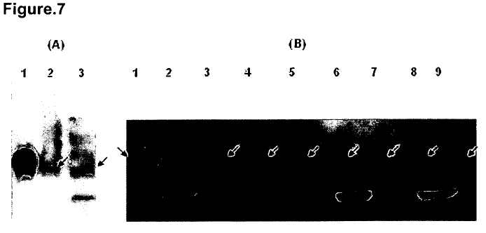

From a total of 71 littermates, 15 were positive for the presence of the transgene (FIG. 4), 3 males and 12 females. DNA hybridization techniques allowed us to corroborate PCR results and to determine the number of copies integrated into transgenic mice genome (Sambrook et al (1982). Molecular cloning-a laboratory manual. 2nd Ed. Cold Spring Harbor Laboratory Press). Homozygous animals were obtained by crossing. It was determined by DNA hybridization techniques that the progeny of homozygous mice carry one or two copies of the transgene, and that it is transmitted to the next generation according to Mendel genetic laws. Human transgene expression is ubiquitous in transgenic mice inside and outside Central Nervous System, according to Northern (FIG. 6) and Western blot (FIG. 7) results.

### Example 2

### Functional Testing of Transgenic Mouse Line F066 by Rotarod

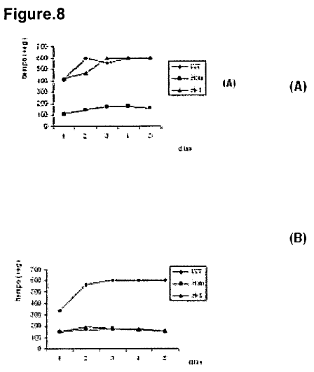

This example describes functional characterization of SCA2 transgenic mouse lines by Rotarod. Motor coordination performances of wild type, homozygous and heterozygous mice were tested using Basile Rotarod treadmills. The duration of the test was 5 days with 4 individual trials Mice were placed on an accelerating rod with a rotating speed from 4 to 40 r.p.m. for 300 seg and with a constant speed of 40 rpm until 600 seg with a resting time of 15 min, between each test. An example of mice performance on the test is represented in FIG. 8. The graph shows average performance on the Rotarod apparatus of four trials each day on five consecutive days. Transgenic mice fall early from Rotarod than wild type mice, showing motor deficit. Differences were significant for homozygous at 8 weeks, and for heterozygous mice at 16 weeks. Rotarod has been used extensively to assess functional impairment in SCA2 transgenic animals. First it was used to evaluate the 15 F0 positive by PCR animals, then it was used to evaluate the F1, F2 and homozygous progenies.

### Example 3

### Evaluation of Homozygous Animals Derived From the F066 Mice

The homozygous mice derived from the FO66 mice, were obtained by matting the founder F066 mice and a transgenic F1 animal. The homozygous character was evaluated by matting with wild type animals. The progenies derived from the matting produced 100% heterozygous animals. The result was demonstrated by PCR analysis of F3 descendents. After analysis of DNA by PCR all the animals were positive for the presence of the ADNc-SCA2-75CAG transgene. The motor deficit was also demonstrated in all animals derived from a homozygous parent. The progenies derived from four generations keep the same morphological, reproductive and conduct phenotype as the parental animals.

### Example 4

### Footprinting Analysis of Transgenic Line F066

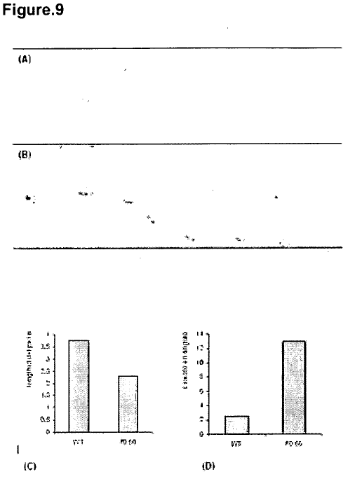

Mouse was allowed to run through a dark 30 cm long, 10 cm wide and 6 cm height tunnel. To measure stride length, the back foots were painted with black aqueous ink. The direction of foot printing was also evaluated. The angle difference between two consecutive steps was calculated. The absolute value of angles was expressed as the sum of angles differences divided by the number of steps. The total stride length value obtained from each animal was an average of the stride length from the total number of steps produced by each leg. Footprint analysis showed progressive reduction in stride length for the transgenic animal compared with the wild type. A typical result is shown in FIG. 9.

### Example 5

### Clasping Evaluation of the F066 Transgenic Line

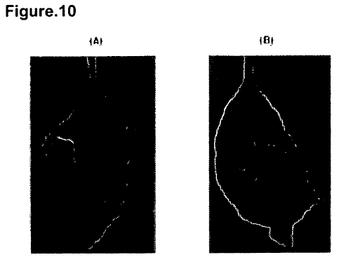

Mice with neurodegenerative phenotypes have a tendency to fold their hindlegs when held by the tail for at least one minute. For the clasping test, mice were held by the tail for one minute. FIG. 10 shows the clasping observed in transgenic animals derived from the F066 mice. No clasping was observed in wild type animals from the same age.

### Example 6

### Anatomical Changes Observed in the Transgenic Line FO66

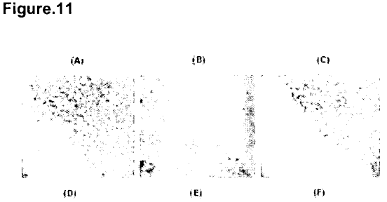

This example describes morphological characterization of SCA2 transgenic mouse lines. Transgenic and control wild type mice from the same age were sacrificed. Brains were removed and embedded in paraffin. Tissue sections were stained with cresyl violet to evaluate the anatomical differences between tissues. Transgenic F066 animals showed a gradual Purkinje cell loss (FIG. 11, B y C). To investigate morphological changes in Purkinje cells, calbindin-28K immunoreactivity in tissue sections from heterozygous animals was compared with that from wild-type mice. Calbindin-28K is a protein specifically expressed in cytoplasm and dendritic processes of cerebellar Purkinje cells. Changes in calbindin-28K labeling were seen in young animals but became more pronounced with age. Most Purkinje cells lost calbindin-28K immunoreactivity in transgenic mice from line F066. Loss of the dendritic arbour was also observed closely followed by a loss in Purkinje cell number (FIG. 11, E y F). These results show morphological neurodegenerative phenotypes are exhibited in transgenic mice expressing human SCA2 having a 75 polyglutamine repeat under the control of the human SCA2 promoter.

1. A transgenic mouse, containing integrated in the genome a DNA sequence identified in the sequence listing as SEQ. ID. NO: 1, comprising a polyglutamine tract of about 75 CAG in the first exon of the ataxin-2 polypeptide, said sequence operatively linked to a SCA2 human promoter that reproduce the expression pattern observed in human patients.

2. The transgenic mouse of claim 1, wherein said mouse is homozygous for said transgene.

3. The transgenic mouse of claim 1, wherein said mouse is heterozygous for said transgene.

4. A progeny of the transgenic mouse of claim 1, characterized by the same morphological, reproductive and conduct characteristics as the parental animals.

5. The use of the transgenic animal of claims 1, for the generation of cellular lines containing as part of their genome the sequence identified as SEQ. ID. NO: 1, with an expansion of 75 CAG in the first exon of the SCA2 gene, said sequence operatively linked to a SCA2 human promoter.

6. Cellular lines according to claim 5, containing as part of their genome the sequence identified as SEQ. ID. NO: 1, with an expansion of 75 CAG in the first exon of the SCA2 gene, said sequence operatively linked to a SCA2 human promoter.

7. The use of the progeny of the transgenic mouse of claim 4 for the generation of cellular lines containing as part of their genome the sequence identified as SEQ. ID. NO: 1, with an expansion of 75 CAG in the first exon of the SCA2 gene, said sequence operatively linked to a SCA2 human promoter.

8. Cellular lines according to claim 7, containing as part of their genome the sequence identified as SEQ. ID. NO: 1, with an expansion of 75 CAG in the first exon of the SCA2 gene, said sequence operatively linked to a SCA2 human promoter.
